(19) United States
(12) Patent Application Publication
(10) Pub. No.: US 2020/0170912 A1
(43) Pub. Date: Jun. 4, 2020

(54) COMPOSITION FOR THE CARE OF HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Hamburg (DE)

(73) Assignee: Henkel AG & Co. KGaA, Duesseldorf (DE)

(21) Appl. No.: 16/689,495

(22) Filed: Nov. 20, 2019

(30) Foreign Application Priority Data
Dec. 3, 2018 (DE) 10 2018 220 849.2

(51) Int. Cl. A61K 8/41; A61K 8/31; A61K 8/34; A61Q 5/00

(52) U.S. Cl. CPC A61K 8/416; A61K 8/31; A61K 2800/5426; A61K 8/345; A61Q 5/002; A61K 8/342

(57) ABSTRACT

Hair treatment agents containing
a) from about 0.1 to about 10% by weight of at least one branched and/or linear alkane with 9 to 17 carbon atoms,
b) from about 2 to about 10% by weight of at least one linear or branched, saturated or unsaturated fatty alcohol with 16 to 22 carbon atoms,
c) from about 2 to about 10% by weight of at least one linear or branched, saturated or unsaturated fatty alcohol with 10 to 14 carbon atoms,
d) from about 0.1 to about 10% by weight of at least one cationic surfactant, comprising a quaternary ammonium compound, and
e) from about 1 to about 10% by weight of at least one linear or branched monovalent alcohol with 2 to 8 carbon atoms are particularly suitable
   for the care of hair, in particular for improving
   the wet and/or dry combability,
   the feel of the hair, and
   for improving the ease of spreading.

### CROSS-REFERENCE TO RELATED APPLICATION

This application claims priority to German Patent Application No. 10 2018 220 849.2, filed Dec. 3, 2018, which is incorporated herein by reference in its entirety.

### TECHNICAL FIELD

The application describes hair treatment agents based on an active substance combination of specific alkanes, a mixture of specific fatty alcohols, and cationic surfactants and alcohols. The application also describes the use of these agents for hair care.

### BACKGROUND

Besides natural environmental influences, human hair is also exposed to a series of other, particularly cosmetic, stresses. These hair-straining stresses include, for example, the coloring of hair and its deformation, for example by perming. Cosmetic hair care products are used to reduce the negative effects of the (environmental) influences impairing the hair structure, while also retaining and improving the natural hair structure. Silicone-organic compounds are an essential active substance in many of these cosmetic agents, in particular silicones such as trisiloxanes, which are exemplified by hair-caring properties. The disadvantages of these silicones are the reduced penetration of active and auxiliary substances into the hair and the complication of hair styling, each caused by the moistening of the hair surface. Furthermore, for reasons of sustainability, efforts must be made to use as high a proportion of biodegradable active substances as possible in cosmetic products. The preparation of low-silicone or silicone-free care products is therefore a relevant problem in the field of hair cosmetics.

In the past, a series of “silicone substitutes” have been proposed as care substances in hair treatment agents, for example linear and/or branched paraffins:

For example, cosmetic compositions are described in WO 2010/115973A1 (Biosynthis) which contain mixtures of linear C₈-C₁₂ alkanes and C₁₄-C₂₄ alkanes in addition to further constituents.
EP 413417B1 and EP 1284712B1 relate to hair care shampoos and hair conditioners which contain isoparaffins in addition to further constituents.

Nevertheless, there is still a need for hair care agents—which can be easily and quickly rinsed out as appropriate—and which provide a conditioning advantage and improve the optical appearance of hair treated with them.

### BRIEF SUMMARY

Hair treatment agents and methods of using the same are provided. In an exemplary embodiment, a hair treatment agent includes, in relation to its total weight, parts a) through e). Part a) is from about 0.1 to about 10% of the hair treatment agent, and part a) includes at least one branched and/or linear alkane with 9 to 17 carbon atoms. Part b) is from about 2 to about 10% of the hair treatment agent, and includes at least one linear or branched, saturated or unsaturated fatty alcohol with 16 to 22 carbon atoms. Part c) is from about 2 to about 10% of the hair treatment agent, and includes at least one linear or branched, saturated or unsaturated fatty alcohol with 10 to 14 carbon atoms, while part d) is from about 0.1 to about 10% of the hair treatment agent and includes at least one cationic surfactant with a quaternary ammonium compound. Part e) is from about 1 to about 10% of the hair treatment agent and includes at least one linear or branched monovalent alcohol with 2 to 8 carbon atoms.

A method of using a hair treatment agent is provided in another embodiment. The method includes applying the hair treatment agent to hair. The hair treatment agent includes, based on a total weight of the hair treatment agent: a) from about 0.1 to about 10% of at least one branched and/or linear alkane with 9 to 17 carbon atoms; b) from about 2 to about 10% of at least one linear or branched, saturated or unsaturated fatty alcohol with 16 to 22 carbon atoms; c) from about 2 to about 10% of at least one linear or branched, saturated or unsaturated fatty alcohol with 10 to 14 carbon atoms; d) from about 0.1 to about 10% of at least one cationic surfactant with a quaternary ammonium compound; and e) from about 1 to about 10% of at least one linear or branched monovalent alcohol with 2 to 8 carbon atoms.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has been found that, as a result of the combination of linear and/or branched alkanes with a mixture of specific fatty alcohols, cationic surfactants and alcohols, hair care agents having an excellent care effect can be obtained.

The use of these agents leads to an improvement of the feel of the hair and also to an improvement in the detangling and ease of combing of hair. In addition, these hair care agents have an excellent ease of spreading, which leads to an improved suitability for distribution over the hair fibers from the root to the tip, and thus leads to an overall greater care effect even with relatively short application times.

A first subject of this application is a hair treatment agent which—in relation to its total weight—contains
a) from about 0.1 to about 10% by weight of at least one branched and/or linear alkane with 9 to about 17 carbon atoms,
b) from about 2 to about 10% by weight of at least one linear or branched, saturated or unsaturated fatty alcohol with from about 16 to about 22 carbon atoms,
c) from about 2 to about 10% by weight of at least one linear or branched, saturated or unsaturated fatty alcohol with from about 10 to about 14 carbon atoms,
d) from about 0.1 to about 10% by weight of at least one cationic surfactant, comprising a quaternary ammonium compound, and
e) from about 1 to about 10% by weight of at least one linear or branched monovalent alcohol with 2 to 8 carbon atoms.

Hair treatment agents in the sense of the present disclosure are, for example, hair dyes, bleaching agents, hair shampoos, hair conditioners, conditioning shampoos, hairsprays, hair rinses, hair masks, hair packs, hair tonics, perm fixing solutions, coloring shampoos, hair setting products, hair smoothing products, hairstyling preparations, setting lotions, styling mousses, hair gels, hair wax, or combinations thereof.

The hair treatment agents as contemplated herein are preferably hair conditioners, conditioning shampoos, hair rinses, hair masks, hair packs or hair tonics, and particularly preferably are conventional hair conditioners, such as hair rinses, hair masks and hair packs.

O/W, W/O and W/O/W emulsions in the form or creams or gels, but also foaming solutions containing active cleansing substances, such as shampoos, foam aerosols or other preparations which are suitable in particular for application to the hair, are particularly suitable as cosmetic carriers. However, it is also conceivable to integrate the active substances a) to e) in a formulation provided in powder or tablet form which is dissolved in water prior to use.

In a particularly preferred embodiment the cosmetic carrier is aqueous.

An aqueous cosmetic carrier in the sense of the present disclosure means that the hair treatment agents preferably contain at least about 50% by weight, more preferably at least about 60% by weight, and particularly preferably at least about 70% by weight water (in relation to the total weight of the hair treatment agents).

As first essential ingredient a) the hair treatment agents as contemplated herein contain from about 0.1 to about 10% by weight of at least one branched and/or linear alkane with 9 to about 17 carbon atoms, wherein the specified amounts relate to the total weight of the hair treatment agents.

It has been found that, in particular, medium-chain, branched and/or linear alkanes a) can be incorporated without difficulty into hair care agents containing fatty substances and cationic compounds and significantly improve the ease with which the hair care agents can be distributed over the hair.

Compared to silicones in corresponding hair treatment agents, medium-chain branched and/or linear alkanes a) have an approximately equivalent hair-caring effect (in particular sensory properties and ease of combing).

Suitable linear alkanes ai) are understood to be n-nonane, n-decane, n-undecane, n-dodecane, n-tridecane, n-tetradecane, n-pentadecane, n-hexadecane, n-heptadecane and mixtures of these linear alkanes.

Particularly preferred are n-decane, n-undecane, n-dodecane, n-tridecane and/or n-tetradecane. Particularly preferred are n-undecane, n-dodecane, n-tridecane and mixtures of these alkanes which contain one or more of these alkanes in an amount of at least about 50% by weight, preferably more than about 60% by weight, more preferably more than about 70% by weight, and in particular more than about 80% by weight.

Two or more linear alkanes ai) are preferably used as mixtures.

Particularly preferred mixtures of medium-chain linear alkanes ai) are understood to be mixtures containing C₁₂ alkane (dodecane) in an amount of at least about 50% by weight.

Particularly preferred mixtures of medium-chain linear alkanes ai) also comprise a mixture of undecane and tridecane in an amount of at least about 50% by weight.

Alkanes exclusively from plant sources are preferably used in the agents as contemplated herein as mixtures of linear alkanes ai).

Examples of particularly suitable mixtures of linear alkanes ai) of plant origin are the commercially available products Parafol® 12-97 (INCI name: Dodecane) and Cetiol® Ultimate (INCI name: Undecane; Tridecane).

In a first preferred embodiment the hair treatment agents as contemplated herein contain n-undecane, n-dodecane, n-tridecane and mixtures thereof.

The linear alkanes ai) or mixtures thereof can be used in the hair treatment agents as contemplated herein preferably in a weight proportion in each case of from about 0.1 to about 10% by weight (more preferably from about 0.2 to about 8% by weight, particularly preferably from about 0.3 to about 7% by weight, and in particular from about 0.5 to about 6% by weight) in the total weight of the hair treatment agents.

Isoparaffins with 9 to about 17 carbon atoms aii) are suitable as silicone substitutes in hair care agents on account of their ability to impart shine and softness on the hair without weighing down the hair or making it greasy in the case of regular use.

Suitable branched alkanes (isoparaffins) with 9 to about 17 carbon atoms aii) are preferably understood to be isodecane, isoundecane, isododecane, isotridecane, isotetradecane and mixtures of these isoparaffins.

Individual isoparaffins of this kind or mixtures of two or more isoparaffins can be used. The use of isoparaffins from the group of isoundecane, isododecane and isotridecane have proven to be particularly advantageous for the cosmetic effect in the hair treatment agents as contemplated herein.

In a second preferred embodiment the hair treatment agents as contemplated herein contain isoundecane, isododecane, isotridecane and mixtures thereof, particularly preferably an isoparaffin known under the INCI name Isododecane.

The weight proportion of the isoundecane, isododecane, isotridecane in the total weight of all isoparaffins aii) used in the hair treatment agents as contemplated herein is preferably > about 60% by weight, more preferably > about 70% by weight, particularly preferably > about 80% by weight, and in particular preferably > about 90% by weight.

Examples of particularly suitable isoparaffins aii) are the commercially available products Purolan® IDD (INCI name: Isododecane), Pioneer® 2094 and KC Solvent® 130 (INCI name: Isoundecane, Isododecane).

The isoparaffin(s) aii) can be used in the hair treatment agents as contemplated herein preferably in a weight proportion in each case of from about 0.1 to about 10% by weight (more preferably from about 0.25 to about 8% by weight, particularly preferably from about 0.5 to about 7% by weight, and in particular from about 1.0 to about 6% by weight) in the total weight of the hair treatment agents.

In a further preferred embodiment, hair treatment agents as contemplated herein contain, as component a), a mixture of linear alkanes ai) with 9 to about 17 carbon atoms and isoparaffins aii) with 9 to about 17 carbon atoms in the aforementioned amounts.

As essential components b) and c), the hair treatment agents as contemplated herein each contain from about 2 to about 10% by weight of at least one fatty alcohol of a certain chain length.

It has been found that a mixture of medium-chain and longer-chain fatty alcohols in the hair treatment agents as contemplated herein leads to a significant increase in the care parameters. The hair treatment agents as contemplated herein in emulsion form appear to be richer, creamier and softer, which results in very good application alongside the property of being easily rinsed out.

Suitable fatty alcohols b) are understood to be linear or branched, saturated or unsaturated fatty alcohols with about 16 to about 22 carbon atoms.

Linear, saturated fatty alcohols with about 16 to about 18 carbon atoms are preferably used in the hair treatment agents as contemplated herein, particularly preferably cetyl alcohol, cetearyl alcohol, stearyl alcohol, and in particular cetearyl alcohol.

The usage amount of the at least one fatty alcohol b), preferably cetyl alcohol, cetearyl alcohol and/or stearyl alcohol, in the hair treatment agents as contemplated herein is preferably from about 2.5 to about 8% by weight, particularly preferably from about 3 to about 7% by weight, and in particular from about 3.5 to about 6% by weight (in relation to the total weight of the hair treatment agent).

In a third preferred embodiment the hair treatment agents as contemplated herein contain, as component b), cetyl alcohol, cetearyl alcohol, stearyl alcohol and mixtures thereof, preferably cetearyl alcohol.

Suitable fatty alcohols c) are understood to be linear or branched, saturated or unsaturated fatty alcohols with about 10 to about 14 carbon atoms.

Linear, saturated fatty alcohols with about 12 to about 14 carbon atoms are preferably used in the hair treatment agents as contemplated herein, particularly preferably lauryl alcohol, tridecyl alcohol, myristyl alcohol, and in particular myristyl alcohol.

The usage amount of the at least one fatty alcohol c), preferably lauryl alcohol, tridecyl alcohol, and/or myristyl alcohol, in the hair treatment agents as contemplated herein is preferably from about 2.5 to about 8% by weight, particularly preferably from about 3 to about 7% by weight, and in particular from about 3.5 to about 6% by weight (in relation to the total weight of the hair treatment agent).

In a third preferred embodiment the hair treatment agents as contemplated herein contain, as component c), lauryl alcohol, tridecyl alcohol, myristyl alcohol and mixtures thereof, preferably myristyl alcohol.

As a fourth essential constituent of the active substance mixture as contemplated herein, the hair treatment agents contain (in relation to their total weight) from about 0.1 to about 10% by weight of at least one cationic surfactant, comprising at least one quaternary ammonium compound d).

Suitable quaternary ammonium compounds d) are understood to be compounds of the following formula (I),

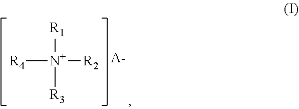

in which
the groups R₁, R₂, R₃ and R₄, each independently of one another, stand for hydrogen, a methyl group, a phenyl group, a benzyl group or for a saturated, branched or unbranched alkyl group with a chain length of from 8 to about 30 carbon atoms, which can be substituted optionally with one or more hydroxy groups,
   with the provision that at least one of the groups R₁, R₂, R₃ and R₄ does not stand for hydrogen, and
A⁻ stands for a physiologically acceptable anion, for example for a halide such as chloride or bromide, and for a methosulfate.

Compounds of formula (I) in which one of the groups R₁, R₂, R₃ and R₄ stand for a saturated branched or unbranched alkyl group with a chain length of from about 16 to about 24 carbon atoms and the other three groups stand for methyl groups are particularly preferred.

Examples of particularly preferred compounds of formula (I) are lauryl trimethyl ammonium chloride, cetyl trimethyl ammonium chloride, cetyl trimethyl ammonium bromide, cetyl trimethyl ammonium methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide, behenyl trimethyl ammonium methosulfate.

Cetyl trimethyl ammonium chloride and/or behenyl trimethyl ammonium chloride are/is preferred in particular.

In a fourth preferred embodiment the hair treatment agents as contemplated herein contain, as component d), at least one C₁₆-C₂₄ alkyl trimethyl ammonium salt, preferably a cetrimonium salt and/or a behentrimonium salt.

The usage amount of the at least one quaternary ammonium compound d), preferably of compounds according to formula (I) and in particular of cetrimonium salts and/or behentrimonium salts, in the hair treatment agents as contemplated herein is preferably from about 0.5 to about 8% by weight, particularly preferably from about 1.0 to about 7% by weight, and in particular from about 1.5 to about 6% by weight (in relation to the total weight of the hair treatment agent).

As a fifth essential constituent of the active substance mixture as contemplated herein, the hair treatment agents contain (in relation to their total weight) from about 1.0 to about 10% by weight of at least one linear or branched monovalent alcohol with 2 to 8 carbon atoms e).

It has been found that with addition of alcohols e) the emulsion picture of the hair treatment agents as contemplated herein changes positively insofar as the emulsion achieves a finer droplet size and therefore the ease of distribution of the agents over the hair is improved.

Suitable monovalent alcohols e) are preferably understood to be ethanol and/or propanol, and in particular ethanol.

In a fifth preferred embodiment the hair treatment agents as contemplated herein contain, as component e), ethanol and/or propanol, and in particular ethanol.

The usage amount of the at least one alcohol e), preferably ethanol and/or propanol, and in particular ethanol, in the hair treatment agents as contemplated herein is preferably from about 1.5 to about 8% by weight, particularly preferably from about 2.0 to about 7% by weight, and in particular from about 2.5 to about 6% by weight (in relation to the total weight of the hair treatment agent).

A sixth particularly preferred embodiment of the hair treatment agents contain—in relation to their total weight—
a) from about 0.1 to about 10% by weight of isoundecane, isododecane, isotridecane and mixtures thereof,
b) from about 2 to about 10% by weight of cetyl alcohol, cetearyl alcohol, stearyl alcohol and mixtures thereof,
c) from about 2 to about 10% by weight of lauryl alcohol, tridecyl alcohol, myristyl alcohol and mixtures thereof,
d) from about 0.1 to about 10% by weight of at least one C₁₆-C₂₄ alkyl trimethyl ammonium salt, and
e) from about 1 to about 10% by weight of ethanol and/or propanol.

Within this embodiment, hair treatment agents are very particularly preferred which contain (in relation to their total weight)
a) from about 0.1 to about 10% by weight, preferably from about 0.25 to about 8% by weight, particularly preferably from about 0.5 to about 7% by weight, and in particular from about 1.0 to about 6% by weight of isoundecane, isododecane, isotridecane and mixtures thereof,
b) from about 2 to about 10% by weight, preferably from about 2.5 to about 8% by weight, particularly preferably from about 3 to about 7% by weight, and in particular from about 3.5 to about 6% by weight of cetearyl alcohol,
c) from about 2 to about 10% by weight, preferably from about 2.5 to about 8% by weight, particularly preferably from about 3 to about 7% by weight, and in particular from about 3.5 to about 6% by weight of myristly alcohol,
d) from about 0.1 to about 10% by weight, preferably from about 0.5 to about 8% by weight, particularly preferably from about 1.0 to about 7% by weight, and in particular from about 1.5 to about 6% by weight of cetrimonium and/or behentrimonium salts, and
e) from about 1 to about 10% by weight, preferably from about 1.5 to about 8.0% by weight, particularly preferably from about 2.0 to about 7% by weight, and in particular from about 2.5 to about 6% by weight of ethanol.

A sixth particularly preferred embodiment of the hair treatment agents contain—in relation to their total weight—
a) from about 0.1 to about 10% by weight of n-undecane, n-dodecane, n-tridecane and mixtures thereof,
b) from about 2 to about 10% by weight of cetyl alcohol, cetearyl alcohol, stearyl alcohol and mixtures thereof,
c) from about 2 to about 10% by weight of lauryl alcohol, tridecyl alcohol, myristyl alcohol and mixtures thereof,
d) from about 0.1 to about 10% by weight of at least one C₁₆-C₂₄ alkyl trimethyl ammonium salt, and
e) from about 1 to about 10% by weight of ethanol and/or propanol.

Within this embodiment, hair treatment agents are very particularly preferred which contain (in relation to their total weight)
a) from about 0.1 to about 10% by weight, preferably from about 0.25 to about 8% by weight, particularly preferably from about 0.5 to about 7% by weight, and in particular from about 1.0 to about 6% by weight of n-undecane, n-dodecane, n-tridecane and mixtures thereof,
b) from about 2 to about 10% by weight, preferably from about 2.5 to about 8% by weight, particularly preferably from about 3 to about 7% by weight, and in particular from about 3.5 to about 6% by weight of cetearyl alcohol,
c) from about 2 to about 10% by weight, preferably from about 2.5 to about 8% by weight, particularly preferably from about 3 to about 7% by weight, and in particular from about 3.5 to about 6% by weight of myristyl alcohol,
d) from about 0.1 to about 10% by weight, preferably from about 0.5 to about 8% by weight, particularly preferably from about 1.0 to about 7% by weight, and in particular from about 1.5 to about 6% by weight of cetrimonium and/or behentrimonium salts, and
e) from about 1 to about 10% by weight, preferably from about 1.5 to about 8.0% by weight, particularly preferably from about 2.0 to about 7% by weight, and in particular from about 2.5 to about 6% by weight of ethanol.

To further increase the hair-caring properties and/or to further increase the excellent ease of spreading of the hair treatment agents as contemplated herein, it may additionally be of advantage if further active substances are added to them.

Polyols f) are further suitable active substances that may be employed to increase the texture and feel of the hair treatment agents as contemplated herein.

Preferred polyols are C₃-C₁₂ polyols, particularly preferably C₃-C₆ polyols that are liquid under normal conditions, such as 1,2- and 1,3-propylene glycols, butylene, pentylene and/or hexylene glycols, sorbitol, mannitol, glycerol, and mixtures thereof.

Vicinal C₃-C₆ diols, and in particular 1,2-propanediol, are preferred in particular on account of their availability, their smell, which is suitable for use in cosmetic agents, and their environmental compatibility.

The at least one polyol f) can be used in the hair treatment agents as contemplated herein preferably in an amount of from about 0.1 to about 6% by weight, more preferably from about 0.25 to about 5% by weight, particularly preferably from about 0.5 to about 4% by weight, and in particular from about 0.75 to about 3% by weight, wherein the specified amounts relate to the total weight of the hair treatment agents.

In a further preferred embodiment the hair treatment agents as contemplated herein additionally contain from about 0.1 to about 6% by weight, more preferably from about 0.25 to about 5% by weight, particularly preferably from about 0.5 to about 4% by weight, and in particular from about 0.75 to about 3% by weight of at least one C₃-C₁₂ polyol, preferably a C₃-C₆ polyol and in particular a vicinal C₃-C₆ diol.

Within this embodiment, hair treatment agents as contemplated herein which contain 1,2-propanediol as component f) are very particularly preferred.

In a further preferred embodiment the hair treatment agents as contemplated herein can additionally contain, as further hair-care active substances, from about 0.1 to about 6% by weight, preferably from about 0.25 to about 5% by weight, particularly preferably from about 0.5 to about 4% by weight and in particular from about 0.75 to about 3% by weight

i) of at least one—optionally cationized—amidoamine g) and/or
ii) at least one cationic polymer h).

Amidoamines and/or cationized amidoamines g) can increase, even further, the care effect of hair treatment agents containing alkanes a), fatty alcohols b)+c), cationic care substances d), and alcohols e) by imparting more shine and smoothness to the hair.

In addition, amidoamines and/or cationized amidoamines g) contribute to an increase of the viscosity of the hair treatment agents, so that it is possible to dispense with the use of synthetic thickeners, such as acrylic acid (derivative) polymers.

Suitable amidoamines and/or cationized amidoamines g) are understood, for example, to be compounds of the following formula (II)

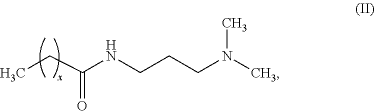

in which
x stands for the numbers 18, 19, 20, 21, 22, 23 or 24.
Compounds of formula (II) with x=20 may be particularly preferred.

Preferred compounds according to formula (II) are commercially available under the INCI name Brassicamidopropyl Dimethylamine, for example under the commercial names Kerabase® LC or ProCondition® 22 from the company Inolex Personal Care Ingredients.

Furthermore, amidoamines and/or cationized amidoamines g) that are suitable as contemplated herein can be selected from compounds of the following formula (III)

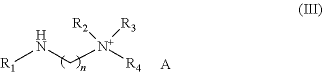

in which
R₁ means an acyl or alkyl group with 6 to 30 C atoms, which can be branched or unbranched, saturated or unsaturated, and wherein the acyl group and/or the alkyl group can contain at least one OH group,
R₂, R₃ and R₄ each independently of one another can stand for
   (i) hydrogen,
   (ii) an alkyl group with 1 to 4 C atoms, which can be the same or different, saturated or unsaturated, or
   (iii) a branched or unbranched hydroxyalkyl group with 1 to 4 carbon atoms, which can be substituted with at least one and at most three hydroxy groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl groups,
A means a physiologically acceptable anion, and
n stands for an integer between 1 and about 10.

For example, halide ions, sulfate ions, phosphate ions, methosulfate ions and organic ions, such as lactate, citrate, tartrate and acetate ions can be considered as physiologically acceptable counterions A. Methosulfates and halide ions, in particular chloride, are preferred.

A hair treatment agent in which the amidoamine and/or the quaternized amidoamine according to general formula (III) is an amidoamine and/or a quaternized amidoamine in which R₁ means a branched or unbranched, saturated or unsaturated acyl group with 6 to 30 C atoms, which can contain at least one OH group, is preferred. Preferred here is a fatty acid group from oils and waxes, in particular from natural oils and waxes. Examples include lanolin, beeswax or candelilla wax.

Amidoamines and/or quaternized amidoamines in which R₂, R₃ and/or R₄ in formula (III) mean a group according to general formula CH₂CH₂OR₅, in which R₅ can have the meaning of alkyl groups with 1 to 4 carbon atoms, hydroxyethyl or hydrogen, are also preferred. The preferred size of n in the general formula (III) is an integer between 2 and 5.

The alkylamidoamines g) according to formula (III) can be present as such and can also be converted by protonation in an appropriately acidic solution into a quaternary compound in the hair treatment agent.

Examples of preferred amidoamines g) as contemplated herein and according to formula (III) are the compounds known under the INCI names Lauramidopropyl Dimethylamine (for example Mackine® 801), Lauramidopropyl Dimethylamine Propionate, Stearamidopropyl Dimethylamine (for example Adogen® S18V or Tego® Amid S 18 or Incromine® SB), Myristamidopropyl Dimethylamine (for example Schercodine® M), Stearamidoethyl Diethylamine (for example Lexamine® 22), Stearamidoethyl Diethylamine Phosphate, Cocamidopropyl Dimethylamine (for example Mackine® 101), Ricinolamidopropyl Dimethylamine (for example Mackine® 201), Isostearamidopropyl Dimethylamine (for example Mackine® 401), Oleamidopropyl Dimethylamine (for example Mackine® 501), Behenamidopropyl Dimethylamine (for example Mackine® 601, Incromine® BD), Cocamidopropyl Dimethylamine Propionate (for example Mackalene® 117), Cocamidopropyl Dimethylamine Lactate (for example Mackalene® 116), Ricinoleamidopropyl Dimethylamine Lactate (for example Mackalene® 216), Stearamidopropyl Dimethylamine Lactate (Mackalene® 316), Behenamidopropyl Dimethylamine Lactate (for example Mackalene® 616), Sunflowerseedamidopropyl Dimethylamine Lactate (for example Mackalene® 1216), Palmamidopropyl Dimethylamine, Palmamidopropyl Dimethylamine Lactate, Palmamidopropyl Dimethylamine Propionate, Oleamidopropyl Dimethylamine Glycolate, and Oleamidopropyl Dimethylamine Lactate.

Examples of permanent cationic amidoamines are the compounds known under the INCI names Quaternium-33 (for example Swanol®, Lanoquat® DES-50), Behenamidopropyl Ethyldimonium Ethosulfate (for example Schercoquat® BAS), Behenamidopropyl PG-Dimonium Chloride (for example Lexquat® AMG-BEO), Oleamidopropyl Ethyldimonium Ethosulfate, Oleamidopropyl PG-Dimonium Chloride (for example Lexquat® AMG-O), Cocamidopropyl Ethyldimonium Ethosulfate (for example Schercoquat® CAS), Cocamidopropyltrimoniumchloride (for example Empigen® CSC), Ricinoleamidopropylethyldimonium Ethosulfate, Rinoleamidopropyltrimoniumchloride, Ricinoleamidopropyltrimoniummethosulfate (for example Rewoquat® RTM 50), Stearamidopropyl Ethyldimonium Ethosulfate (for example Schercoquat® SAS), Stearamidopropyl Trimonium Methosulfate (for example Catagene® SA-70), Undecyleneamidopropyltrimonium Methosulfate (for example Rewoquat® UTM 50), Lauramidopropyl PG-Dimonium Chloride, and Canolamidopropyl Ethyldimonium Ethosulfate (z. B. Schercoquat® COAS).

Preferred amidoamines and/or cationized amidoamines g) according to formula (III) are the compounds known under the INCI names Lauramidopropyl Dimethylamine, Myristamidopropyl Dimethylamine, Stearamidopropyl Dimethylamine, Cocamidopropyl Dimethylamine, Ricinolamidopropyl Dimethylamine, Isostearamidopropyl Dimethylamine, Oleamidopropyl Dimethylamine, Behenamidopropyl Dimethylamine, Palmamidopropyl Dimethylamine, Quaternium-33, Behenamidopropyl Ethyldimonium Ethosulfate, Oleamidopropyl Ethyldimonium Ethosulfate, Cocamidopropyltrimoniumchloride, Rinoleamidopropyltrimoniumchloride, Stearamidopropyl Trimonium Methosulfate, and mixtures hereof.

Particularly preferred are Stearamidopropyl Dimethylamine, Cocamidopropyl Dimethylamine, Ricinolamidopropyl Dimethylamine, Isostearamidopropyl Dimethylamine, Oleamidopropyl Dimethylamine, Behenamidopropyl Dimethylamine, Palmamidopropyl Dimethylamine, Quaternium-33 and Behenamidopropyl Ethyldimonium Ethosulfate.

Very particularly preferred are Stearamidopropyl Dimethylamine, Cocamidopropyl Dimethylamine, Isostearamidopropyl Dimethylamine, Behenamidopropyl Dimethylamine and Bis-Ethyl(isostearylimidazoline) Isostearamide. Stearamidopropyl Dimethylamine is most preferred.

In a further preferred embodiment the hair treatment agents as contemplated herein contain, as amidoamine and/or a cationized amidoamine g), at least one of the compounds known under the INCI names Brassicamidopropyl Dimethylamine, Stearamidopropyl Dimethylamine, Cocamidopropyl Dimethylamine, Behenamidopropyl Dimethylamine, Isostearamidopropyl Dimethylamine and Bis-Ethyl(isostearylimidazoline) Isostearamide.

Particularly preferred within this embodiment are Brassicamidopropyl Dimethylamine and/or Stearamidopropyl Dimethylamine.

The aforementioned amidoamines and/or a cationized amidoamines g) can be used individually or in any combinations with one another, wherein they are preferably contained in a total amount of from about 0.1 to about 5.0% by weight, more preferably from about 0.2 to about 4.0% by weight, particularly preferably from about 0.3 to about 3% by weight, and very particularly preferably from about 0.4 to about 2.0% by weight, in each case in relation to the weight of the hair treatment agents as contemplated herein.

Cationic polymers h) which are suitable as contemplated herein can preferably be selected from:
cationic polymers which are derived from natural polymers, such as derivatives of polysaccharides, for example cationic derivatives of cellulose, starch or guar. Cationic polysaccharides have the general formula G-O-B-N⁺RₐR_{b}R_{c} A⁻, wherein
G is an anhydroglucose group, for example starch or cellulose anhydroglucose;
B is a divalent compound group, for example alkylene, oxyalkylene, polyoxyalkylene or hydroxyalkylene;
Rₐ, R_{b} and R_{c} independently of one another are alkyl, aryl, alkylaryl, arylalkyl, alkoxyalkyl or alkoxyaryl, in each case with up to about 18 C atoms, wherein the total number of C atoms in Rₐ, R_{b} and R_{c} is preferably at most about 20;
A⁻ is a standard counteranion, preferably chloride.
   In particular, the compounds known under the INCI name Polyquaternium-10 are suitable cationic celluloses. These are available commercially for example under the name Celquat® or Polymer JR®.

Further suitable cationic polymers derived from natural polymers are cationic guar derivatives, in particular guar hydroxypropyltrimonium salts, such as those available commercially under the commercial names Jaguar® or N-Hance®.
Polymers derived from natural cationic polymers are for example cationic cellulose and/or hydrophobically modified cationic cellulose. Such hydrophobically modified cationic celluloses are commercially obtainable with a different degree of substitution, cationic charge density, nitrogen content and molecular weights. In particular, the following modified cationic celluloses are highly preferred as contemplated herein:
Polyquaternium-67; for example commercially available under the names Polymer® SL or Polymer® SK (Amerchol).
A further highly preferred cellulose is offered under the commercial name Mirustyle® CP from the company Croda. This is a trimonium and cocodimonium hydroxyethylcellulose as derivatized cellulose with the INCI name Polyquaternium-72. Polyquaternium-72 can be used both in solid form and already pre-dissolved in an aqueous solution.
A further highly preferred cationic synthetic polymer is a homopolymer that is crosslinked if desired: poly(methacryloyloxyethyltrimethylammonium chloride) with the INCI name Polyquaternium-37. Such products are commercially available for example under the names Rheocare® CTH (Cosmetic Rheologies) and Synthalen® CR (3V Sigma). The homopolymer is preferably used in the form of a non-aqueous polymer dispersion. Such polymer dispersions are commercially available under the names Salcare® SC 95 and Salcare® SC 96.

In a further particularly preferred embodiment the hair treatment agents as contemplated herein contain, as cationic polymer h), at least one of the compounds known under the INCI names Guar Hydroxypropyltrimonium Chloride and Polyquaternium-37.

The at least one cationic polymer h), preferably Guar Hydroxypropyltrimonium Chloride and/or Polyquaternium-37, can be used in the hair treatment agents as contemplated herein preferably in a total amount of from about 0.05 to about 5% by weight, preferably from about 0.1 to about 4.0% by weight, particularly preferably from about 0.15 to about 3.5% by weight, and in particular from about 0.2 to about 3.0% by weight (in relation to the total weight of the hair treatment agent).

Besides the essential components a) to e) and the preferred optional components f) to h), the hair treatment agents as contemplated herein can contain further active and care substances which impart advantageous properties on the agents or further support the advantageous properties of the agents. These include, for example, further cationic surfactants, such as esterquats, ester oils, plant oils, polar oils, vitamins, UV filters, anti-dandruff active substances, protein hydrolyzates, and/or plant extracts.

Suitable esterquats can preferably be selected from compounds of the following formula (IV)

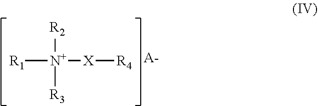

in which the groups R₁, R₂ and R₃, each independently of one another, can be the same or different and can have the following meaning:
a branched or unbranched alkyl group with 1 to 4 carbon atoms, which can contain at least one hydroxyl group, or
a saturated or unsaturated, branched or unbranched, or a cyclic saturated or unsaturated alkyl group with 6 to about 30 carbon atoms, which can contain at least one hydroxyl group, or
an aryl or alkylaryl group, for example phenyl or benzyl,
the group (—X—R₄), with the provision that at most 2 of the groups R₁, R₂ and R₃ can stand for this group, wherein
X stands for:
   1. —(CH₂)ₙ— with n=1 to about 20, preferably n=1 to about 10 and particularly preferably n=1 to 5, or
   2. —(CH₂—CHR₅—O)ₙ— with n=1 to about 200, preferably 1 to about 100, particularly preferably 1 to about 50, and in particular 1 to about 20, and R₅ stands for hydrogen, or a methyl or ethyl group,
   3. a hydroxyalkyl group with one to four carbon atoms, which can be branched or unbranched, and which contains at least one and at most 3 hydroxy groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl groups, and

R₄ stands for:

1. the group R₆-O—CO—, wherein R₆ stands for a saturated or unsaturated, branched or unbranched or a cyclic saturated or unsaturated alkyl group with 6 to about 30 carbon atoms, which can contain at least one hydroxy group, and which optionally also can be oxyethylated with 1 to about 100 ethylene oxide units and/or 1 to about 100 propylene oxide units, or
2. the group R₇-O—CO—, wherein R₇ stands for a saturated or unsaturated, branched or unbranched or a cyclic saturated or unsaturated alkyl group with 6 to about 30 carbon atoms, which can contain at least one hydroxy group, and which optionally also can be oxyethylated with 1 to about 100 ethylene oxide units and/or 1 to about 100 propylene oxide units, and

A⁻ stands for a physiologically compatible organic or inorganic anion, for example a halide ion, such as chloride, bromide, iodide, a sulfate ion of formula RSO₃⁻, wherein R has the meaning of saturated or unsaturated alkyl groups with 1 to 4 carbon atoms, or an organic acid anion, such as for maleate, fumarate, oxalate, tartrate, citrate, lactate or acetate.

Products according to formula (IV) are sold for example under the commercial name Rewoquat®, Stepantex®, Dehyquart®, Armocare® and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these esterquats.

Further esterquats which are suitable as contemplated herein can be selected from compounds of formula (V)

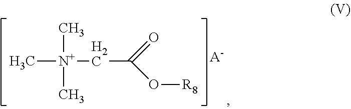

in which the meaning of R₈ corresponds to the meaning of R₇ and in which A⁻ has the same meaning as in formula (IV).

The esterquats according to formula (V) with the commercial names Armocare® VGH-70, Dehyquart® F-75, Dehyquart® L80, Stepantex® VS 90 and Akypoquat® 131 are particularly preferred. Highly preferred among these esterquats are understood to be those referred to according to the INCI naming as Distearoylethyl Hydroxyethylmonium Methosulfate, Dicocoylethyl Hydroxyethylmonium Methosulfate, Dipalmitoylethyldimonium Chloride, and Behenoyl PG Trimonium Chloride.

Esterquats can be used in the hair treatment agents as contemplated herein (in relation to the total weight thereof) preferably in amounts of from about 0.1 to about 5.0% by weight, more preferably from about 0.2 to about 4.0% by weight, particularly preferably from about 0.25 to about 3.0% by weight, and in particular in amounts of from about 0.3 to about 2.5% by weight.

Ester oils that are suitable as contemplated herein are understood to be the esters of single- or multi-base C₆-C₃₀ fatty acids with mono- or polyvalent C₂-C₃₀ fatty alcohols. The monoesters of fatty acids with monovalent alcohols with 2 to about 24 C atoms are preferred.

Examples of fatty acid components used in the esters are caproic acid, caprylic acid, 2-ethylhexanoic acid, isononanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachinic acid, gadoleic acid, behenic acid and erucic acid as well as the technical grade mixtures thereof.

Examples of the fatty alcohol components in the ester oils are isopropyl alcohol, glycerol, caproic alcohol, caprylic alcohol, 2-ethylhexyl alcohol, nonyl alcohol, isononyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as the technical grade mixtures thereof.

Particularly preferred as contemplated herein are isopropyl myristate, isononanoic acid-C16-18-alkyl ester, 2-ethylhexyl palmitate, stearic acid-2-ethylhexyl ester, cetyl oleate, glycerol tricaprylate, coco fatty alcohol-caprinate/-caprylate, n-butyl stearate, oleyl erucate, isopropyl palmitate, oleyl oleate, lauric acid hexylester, di-n-butyl adipate, myristyl myristate, cetearyl isononanoate, isononyl isononanoate and oleic acid decylester and mixtures hereof.

According to a further particularly preferred embodiment, isopropyl myristate and/or isopropyl palmitate are preferred in particular.

The ester oils can optionally also be alkoxylated with ethylene oxide, propylene oxide or mixtures of ethylene oxide and propylene oxide. The alkoxylation can be found at the fatty alcohol part, at the fatty acid part, or at both parts of the ester oils. However, it is preferred as contemplated herein if the fatty alcohol is first alkoxylated and then esterified with fatty acid. These compounds are presented generally in the formula (VI):

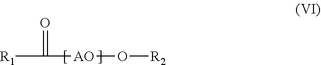

R₁ stands here for a saturated or unsaturated, branched or unbranched, cyclically saturated or cyclically unsaturated acyl group with 6 to about 30 carbon atoms,
AO stands for ethylene oxide, propylene oxide or butylene oxide,
X stands for a number between 1 and about 200, preferably 1 and about 100, particularly preferably between 1 and about 50, very particularly preferably between 1 and about 20, highly preferably between 1 and about 10, and most preferably between 1 and 5,
R₂ stands for a saturated or unsaturated, branched or unbranched, cyclically saturated or cyclically unsaturated alkyl, alkenyl, alkynyl, phenyl or benzyl group with 6 to about 30 carbon atoms.

Examples of fatty acid components used as group R in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachinic acid, gadoleic acid, behenic acid and erucic acid as well as the technical grade mixtures thereof. Examples of the fatty alcohol components as group R2 in the ester oils are benzyl alcohol, isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as the technical grade mixtures thereof.

An alkoxylated ester oil which is preferred as contemplated herein is obtainable for example under the INCI name PPG-3 Benzyl Ether Myristate.

Ester oils are also understood to include:
dicarboxylic acid esters such as di-n-butyladipate, di-(2-ethylhexyl)-adipate, di-(2-ethylhexyl)-succinate and diisotridecylacelate as well as diolesters such as ethylene glycol dioleate, ethylene glycol diisotridecanoate, propylene glycol-di(2-ethylhexanoate), propylene glycol-di-isostearate, propylene glycol-dipelargonate, butanediol-diisostearate, neopentyl glycol dicaprylate, and
symmetrical, asymmetrical or cyclic esters of carbonic acid with fatty alcohols, for example glycerol carbonate or dicaprylyl carbonate (for example Cetiol® CC),
Fatty acid partial glycerides, that is to say monoglycerides, diglycerides and the technical grade mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachinic acid, gadoleic acid, behenic acid and erucic acid as well as the technical grade mixtures thereof. Oleic acid monoglycerides are preferably used.
Natural oils. Examples of such oils are amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, currant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice bran oil, sea buckthorn oil, sea buckthorn oil, sesame oil, shea butter, soybean oil, sunflower oil, grape seed oil, walnut oil, wild rose oil, wheatgerm oil and the liquid components of coconut oil. However, other triglyceride oils, such as the liquid components of bovine tallow, as well as synthetic triglyceride oils are also suitable.

Of course, it is also possible as contemplated herein to use a plurality of ester oils simultaneously. Preferred ester oils are isopropyl myristate, glycerol carbonate, dicaprylyl carbonate, isopropyl palmitate, PPG-3 benzyl ether myristates, cetyloleate, oleylerucate, triglycerides (plant oils) and mixtures of at least two of these ester oils.

In a further preferred embodiment ester oils can be contained in the agents as contemplated herein in a total amount of from about 0.1 to about 10% by weight, more preferably from about 0.1 to about 7.5% by weight, particularly preferably about 0.1 to about 6.0% by weight, highly preferably from about 0.1 to about 5.0% by weight, in relation to the weight of the composition as contemplated herein.

Suitable polar oils are understood to be compounds of the following formula (VII),

R-O(AO)ₙH (VII)

in which
R stands for a linear or crosslinked, saturated or unsaturated alkyl or acyl group with 1 to about 22 carbon atoms, preferably with 3 to about 18 carbon atoms,
AO stands for an oxyalkylene group with 3 to 4, preferably with 3 carbon atoms, and
n stands for the average number of AO units in a range of from 1 to about 100, preferably from 2 to about 50, and in particular 3 to about 20.
   R preferably stands for an alkyl group with 1 to about 22 carbon atoms, preferably with 3 to about 18 carbon atoms.

The polar polyoxyalkylene according to formula (VII) can preferably be selected from one or more of the following compounds:

PPG-2 butyl ether; PPG-3 butyl ether; PPG-4 butyl ether; PPG-5 butyl ether; PPG-9 butyl ether; PPG-12 butyl ether; PPG-14 butyl ether; PPG-15 butyl ether; PPG-16 butyl ether; PPG-17 butyl ether;
PPG-18 butyl ether; PPG-20 butyl ether; PPG-22 butyl ether; PPG-24 butyl ether; PPG-26 butyl ether; PPG-30 butyl ether; PPG-33 butyl ether; PPG-40 butyl ether; PPG-52 butyl ether; PPG-53 butyl ether; PPG-3 caprylyl ether; PPG-4 caprylyl ether; PPG-5 caprylyl ether; PPG-6 caprylyl ether; PPG-10 caprylyl ether; PPG-10 cetyl ether; PPG-20 cetyl ether; PPG-28 cetyl ether; PPG-30 cetyl ether; PPG-50 cetyl ether; PPG-4 lauryl ether; PPG-7 lauryl ether; PPG-10 lauryl ether; PPG-2 methyl ether; PPG-3 methyl ether; PPG-4 methyl ether; PPG-3; myristyl ether PPG-4 myristyl ether; PPG-10 oleyl ether; PPG-20 oleyl ether; PPG-23 oleyl ether; PPG-30 oleyl ether; PPG-37 oleyl ether; PPG-50 oleyl ether; PPG-1 stearyl ether; PPG-15 stearyl ether and mixtures thereof (PPG stands for propylene glycol).

PPG-3 caprylyl ether, PPG-10 lauryl ether and/or PPG-3 myristyl ether are particularly preferred.

In a further preferred embodiment polar oils according to formula (VII) can be contained in the agents as contemplated herein in a total amount of from about 0.1 to about 5% by weight, more preferably from about 0.2 to about 4.5% by weight, particularly preferably from about 0.25 to about 4.0% by weight, highly preferably from about 0.3 to about 3.5% by weight, in relation to the weight of the composition as contemplated herein.

Further positive effects of the hair treatment agents as contemplated herein can be attained if plant extracts (L) are added to them.

As contemplated herein, the extracts from green tea, oak bark, stinging nettle, witch hazel, hops, henna, chamomile, burdock, horsetail, hawthorn, linden blossom, almond, aloe vera, pine needles, horse chestnut, date palm, cinnamon tree, sandalwood, juniper, coconut, mango, apricot, lemon, wheat, kiwi, melon, orange, grapefruit, sage, rosemary, birch, mallow, valerian, lady's smock, wild thyme, yarrow, thyme, lemon balm, Hauhechel, coltsfoot, marshmallow, meristem, ginseng, coffee, cocoa, moringa, ginger root and ayurvedic plant extracts such as Aegle Marmelos (Bilwa), Cyperus rotundus (Nagar Motha), Emblica Officinalis (Amalki) Morida Citrifolia (Ashyuka), Tinospora Cordifolia (Guduchi), Santalum album, (Chan Dana), Crocus sativus (Kumkuma), Cinnamonum Zeylanicum and Nelumbo nucifera (Kamala), grasses such as wheat, barley, rye, oats, spelt, corn, the different varieties of millet (proso millet, finger millet, foxtail millet as examples), sugar cane, rye grass, meadow foxtail, oat grass, bent grass, meadow fescue, moor grass, bamboo, cotton grass, Pennisetum, Andropogonodeae (called Imperata cylindrica and flames grass or Cogon grass), buffalo grass, Spartina, Cynodon, Love grasses, Cymbopogon (lemon grass), Oryzeae (rice), Zizania (wild rice), beach grass, shrubs oats, holcus, dither grasses, bluegrasses, couch grass and Echinacea, in particular Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench and Echinacea pallida (Nutt), all kinds of wine and pericarp of Litchie chinensis are preferred in particular.

Suitable extracts can be obtained from the fruits, seeds, flowers, roots, leaves and/or bark of the above-mentioned plants.

The plant extracts can be used both in pure and diluted form as contemplated herein. If used in diluted form, they usually contain approximately 2-80% by weight of active substance and, as solvent, the extracting agent or extracting agent mixture used for their recovery. Suitable extracting agents are usually water and/or alcohols.

In a preferred embodiment the hair treatment agents as contemplated herein contain aqueous-alcoholic extracts from rice, the fruits of date palms, and/or from the bark of the cinnamon tree.

The plant extract(s) can be used in the hair treatment agents as contemplated herein preferably in total amounts of from about 0.001 to about 1% by weight, wherein the specified amount relates to the weight of the hair treatment agents.

Particularly good hair care results (in particular with regard to the sensory properties such as softness, silkiness and shine as well as combability) can be attained if the hair treatment agents as contemplated herein, besides the above-mentioned alkanes a), fatty alcohols b)+c) as well as optionally besides the ester oils and/or polar oils according to formula (VII), do not contain further fatty phase constituents—in particular no silicones.

In a further preferred embodiment, hair treatment agents as contemplated herein are therefore substantially free from silicones.

The term “substantially free” is understood to mean that the hair treatment agents as contemplated herein preferably contain less than about 0.1% by weight of, more preferably less than about 0.05% by weight of, and in particular no silicones (in relation to the total weight of the hair treatment agents).

The aforementioned values apply here both for freely added silicone and for silicones which optionally may be contained as by-product in commercial products.

A further synergistic active substance as contemplated herein in the compositions as contemplated herein with the active substance combination as contemplated herein is constituted by protein hydrolyzates and/or derivatives (P) thereof.

As contemplated herein protein hydrolyzates of plant and animal or marine or synthetic origin can be used.

Animal protein hydrolyzates are, for example, elastin, collagen, keratin, silk and milk protein hydrolyzates, which can also be present in the form of salts.

Furthermore, preferred plant protein hydrolyzates as contemplated herein are, for example, soy, almond, pea, moringa, potato and wheat protein hydrolyzates. Such products are obtainable for example under the commercial names Gluadin® (BASF), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from the company Laboratoires Sérobiologiques.

Further protein hydrolyzates that are preferred as contemplated herein are of maritime origin. These include, for example, collagen hydrolyzates from fish or algae, and protein hydrolyzates from shells or pearl hydrolyzates. Examples of pearl extracts as contemplated herein are the commercial products Pearl Protein Extract BG® or Crodarom® Pearl.

The protein hydrolyzates (P) can be contained in the hair treatment agents as contemplated herein (in relation to the total weight thereof) in amounts of from about 0.0001% by weight to about 20% by weight, preferably from about 0.05% by weight to about 15% by weight, and very particularly preferably in amounts of from about 0.05% by weight to about 5% by weight.

A further preferred group of ingredients of the compositions as contemplated herein with the active substance combination as contemplated herein are vitamins, provitamins or vitamin precursors.

Here, particularly preferred vitamins, provitamins and vitamin precursors are those assigned to the groups A, B, C, E, F and H.

The group of substances referred to as vitamin A includes retinol (Vitamin A₁) and 3,4-didehydroretinol (Vitamin A₂). β-carotin is the provitamin of retinol. Examples of vitamin A components include, as contemplated herein, for example vitamin A acid and esters thereof, vitamin A aldehyde, and vitamin A alcohol as well as esters thereof, such as the palmitate and acetate. The agents as contemplated herein contain the vitamin A component preferably in amounts of from about 0.05-1% by weight, in relation to the total preparation.

The vitamin B group or the vitamin B complex includes, inter alia:

vitamin B₁ (thiamin)
vitamin B₂ (riboflavin)
vitamin B₃. This often includes the compounds of nicotinic acid and nicotinic acid amide (niacinamide). As contemplated herein the nicotinic acid amide is preferred and can be contained in the agents as contemplated herein preferably in amounts of from about 0.05 to about 1% by weight, in relation to the total agent.

Vitamin B₅ (pantothenic acid, panthenol and pantolactone). In the context of this group, panthenol and/or pantolactone are/is preferably used. Derivatives of panthenol that can be used as contemplated herein are especially the esters and ethers of panthenol, and also cationically derivatized panthenols. Specific representatives are, for example, panthenol triacetate, panthenol monoethyl ether and monoacetate thereof, as well as cationic panthenol derivatives. Pantothenic acid is preferably used in the present disclosure as a derivative in the form of the more stable calcium salts and sodium salts (Ca pantothenate, Na pantothenate).

Vitamin B₆ (pyridoxine and also pyridoxamine and pyridoxal).

The aforesaid compounds of the vitamin B type, in particular vitamin B₃, B₅ and B₆, can be used in the agents as contemplated herein preferably in amounts of from about 0.05-10% by weight, in relation to the total agent. Amounts of from about 0.1-5% by weight are particularly preferred.

Vitamin C (ascorbic acid). Vitamin C can be used in the agents as contemplated herein preferably in amounts of from about 0.1 to about 3% by weight, in relation to the total agent. The use in the form of the palmitic acid ester, the glucosides, or phosphates can be preferred. The use in combination with tocopherols can also be preferred.

Vitamin E (tocopherols, in particular α-tocopherol). Tocopherol and derivatives thereof, in particular including the esters and the acetate, the nicotinate, the phosphate and the succinate, can be used in the agents as contemplated herein preferably in amounts of from about 0.05-1% by weight, in relation to the total agent.

Vitamin F. The term “vitamin F” is usually understood to mean essential fatty acids, in particular linoleic acid, linolenic acid and arachidonic acid.

Vitamin H. The compound (3aS,4S, 6aR)-2-oxohexahydrothienol[3,4-d]-imidazol-4-valeric acid denotes vitamin H, for which the trivial name however (biotin) has become accepted. Biotin can be contained in the agents as contemplated herein preferably in amounts of from about 0.0001 to about 1.0% by weight, in particular in amounts of from about 0.001 to about 0.01% by weight.

The compositions as contemplated herein preferably contain vitamins, provitamins and vitamin precursors from the groups A, B, E and H. Panthenol, pantolactone, pyridoxine and derivatives thereof, as well as nicotinic acid amide and biotin are particularly preferred.

A further preferred group of ingredients in the cosmetic compositions as contemplated herein are the betaines named hereinafter: carnitine, carnitine tartrate, carnitine magnesium citrate, acetyl carnitine, betalaine, 1,1-dimethyl-proline, choline, choline chloride, choline bitartrate, choline dihydrogen citrate and the compound N,N,N-trimethylglycine known in the literature as betaine.

Carnitine, histidine, choline and betaine are preferably used. In a particularly preferred embodiment of the present disclosure L-carnitine tartrate is used as active substance.

A further ingredient is taurine and/or a derivative of taurine. Taurine is understood to mean exclusively 2-aminoethane sulfonic acid and a derivative is understood to refer to the explicitly named derivatives of taurine. The derivatives of taurine are understood to be N-monomethyltaurine, N,N-dimethyltaurine, taurine lysylate, taurine tartrate, taurine ornithate, lysyl taurine and omithyl taurine. Further taurine derivatives in the sense of the present disclosure are taurocholic acid and hypotaurine.

Agents as contemplated herein can contain one or more of the aforementioned active substances, preferably in each case in amounts of from about 0.0001 to about 10.0% by weight, more preferably from about 0.0005 to about 5.0% by weight, particularly preferably from about 0.001 to about 2.0% by weight, and in particular from about 0.001 to about 1.0% by weight (in relation to the total weight of the agent). Taurine and/or one of its derivatives are particularly preferred.

The agents as contemplated herein may additionally contain at least one UV filter. These may be oil-soluble or water-soluble.

Examples of oil-soluble substances are:
3-benzylidenecampher, for example 3-(4-methylbenzylidene)campher;
4-aminobenzoic acid derivatives, preferably 4-(dimethylamino)benzoic acid-2-ethylhexyl esters, 4-(dimethylamino)benzoic acid-2-octyl esters and 4-(dimethylamino)benzoic acid amyl esters;
esters of cinnamic acid, preferably 4-methoxy cinnamic acid-2-ethylhexyl esters, 4-methoxy cinnamic acid propyl esters, 4-methoxy cinnamic acid isoamyl esters, and 2-cyano-3-phenyl-cinnamic acid-2-ethylhexyl esters (octocrylene);
esters of salicylic acid, preferably salicylic acid-2-ethylhexyl esters, salicylic acid-4-isopropylbenzyl esters, and salicylic acid homomenthyl esters;
derivatives of benzophenone, preferably 2-hydroxy-4-methoxybenzophenone, 2-hydroxy-4-methoxy-4′-methylbenzophenone, and 2,2′-dihydroxy-4-methoxybenzophenone;
esters of benzyl malonic acid, preferably 4-methoxybenzyl malonic acid di-2-ethylhexyl esters;
triazine derivatives, such as 2,4,6-trianilino-(p-carbo-2′-ethyl-1′-hexyloxy)-1,3,5-triazine and octyltriazone;
propane-1,3-dione, such as 1-(4-tert.butylphenyl)-3-(4′methoxyphenyl)propane-1,3-dione;
   The following are potential water-soluble substances:
2-phenylbenzimidazole-5-sulfonic acid and the alkali, alkaline earth, ammonium, alkylammonium, alkanolammonium and glucammonium salts;
sulfonic acid derivatives of benzophenonene, preferably 2-hydroxy-4-methoxybenzophenone-5-sulfonic acid and salts thereof;
sulfonic acid derivatives of 3-benzylidene campher, such as 4-(2-oxo-3-bornylidenemethyl)benzene sulfonic acid and 2-methyl-5-(2-oxo-3-bornylidene) sulfonic acid and salts thereof.
   Possible typical UV-A filters are in particular derivatives of benzoylmethane, such as 1-(4′-tert.butylphenyl)-3-(4′-methoxyphenyl)propane-1,3-dione or 1-phenyl-3-(4′-isopropylphenyl)-propane-1,3-dione.

The UV-A and UV-B filters can of course also be used in combination. Besides the mentioned soluble substances, insoluble pigments can also be used for this purpose, in particular finely dispersed metal oxides or salts, for example titanium dioxide, zinc oxide, iron oxide, aluminum oxide, cerium oxide, zirconium oxide, silicates (talc), barium sulfate and zinc stearate. The particles should have a mean diameter of less than about 100 nm, preferably between about 5 and about 50 nm, and in particular between about 15 and about 30 nm. They can have a spherical shape, however particles which have an ellipsoid shape or a shape deviating from the spherical form can also be used.

The hair treatment agents as contemplated herein can also contain further active substances, auxiliaries and additives, for example acidifiers, such as citric acid and lactic acid, dimethylisosorbide and cyclodextrins, dyes for coloring the agent, anti-dandruff active substances, such as piroctone olamine, zinc omadine and climbazol, complexing agents such as EDTA, NTA, β-alanine diacetic acid and phosphonic acids, opacifiers such as Latex, styrene/PVP and styrene/acrylamide copolymers, pearlescent agents such as ethylene glycol mono and distearate and PEG-3 distearate, pigments, stabilizers for hydrogen peroxide and other oxidants, propellants such as propane-butane mixtures, N₂O, dimethyl ether, CO₂ and air, antioxidants, perfume oils, fragrances and aromatic substances.

With regard to further optional components and the used amounts of these components, reference is made expressly to the relevant handbooks known to a person skilled in the art.

For optimal product performance it is also advantageous if the hair treatment agents as contemplated herein have a pH value in the range of from about 2.5 to about 4.5, more preferably from about 3.0 to about 4.5, and in particular from about 3.0 to about 4.0.

A second subject of the present disclosure is the cosmetic use of the hair treatment agent as contemplated herein
for the care of hair, in particular for improving
the wet and/or dry combability,
the feel of the hair, and
for improving the ease of spreading.

That which has been said with regard to the agents as contemplated herein applies, mutatis mutandis, with regard to further preferred embodiments of the use as contemplated herein.

The following examples are intended to explain the subject matter of the present disclosure without limiting it.

### EXAMPLES

### 1) Practical Examples

The following hair treatment agents were prepared as contemplated herein (the stated values are in [% by weight]):

| | 1 | 2 | 3 | 4 | 5 |
|---|---|---|---|---|---|
| Isoundecane, isododecane and/or | 4.0 | | | | |
| isotridecane | | | | | |
| n-undecane, n-dodecane and/or n- | | 4.5 | | | |
| tridecane | | | | | |
| Purolan ®¹ IDD | | | 2.0 | | 4.0 |
| Cetiol Ultimate ®² | | | 2.0 | 4.5 | |
| C₁₆₋₂₂ fatty alcohol | 4.0 | 4.0 | 4.0 | | |
| C₁₀₋₁₄ fatty alcohol | 4.0 | 4.0 | 4.0 | | |
| Cetearyl alcohol | | | 4.0 | 4.0 | 4.0 |
| Nacol ®³ 14-98 | | | 4.0 | 4.0 | 4.0 |
| C16-C24 alkyl trimethyl ammonium | 2.0 | 2.0 | 2.0 | | |
| chloride | | | | | |
| Cetrimonium chloride and/or | | | | 1.75 | 1.75 |
| behentrimonium chloride | | | | | |
| Ethanol | 4.0 | 4.0 | 4.0 | 3.75 | 3.75 |
| 1,2-propanediol | 1.0 | | 1.0 | 1.0 | 1.0 |
| Guar Hydroxypropyltrimonium | 0.1 | 0.1 | | | 0.1 |
| Chloride | | | | | |
| Stearamidopropyl Dimethylamine | 0.8 | 0.8 | 0.8 | | 0.8 |
| Cosmedia Triple ®⁴ | | | 0.4 | | 0.4 |
| Softcare ®⁵ GP-1 | | | | | 1.0 |
| Perfume | 0.35 | 0.35 | 0.35 | 0.35 | 0.35 |
| Water, preservatives, pH adjusters | to 100 | to 100 | to 100 | to 100 | to 100 |

¹INCI name: Isododecane (Lanxess)
²INCI name: Undecane, Tridecane (BASF)
³INCI name: Myristyl Alcohol (Sasol)
⁴INCI name: Polyquaternium-37, Dicaprylyl Carbonate, Lauryl Glucoside (BASF)
⁵INCI name: PPG-3 Caprylyl Ether (KAO)

The following commercial products were used:

### 2) Proof of Efficacy

The following agents A (market standard) and B (composition as contemplated herein) were tested against one another with regard to the hair care parameter of wet combability:

| | A | B |
|---|---|---|
| Isopropyl Myristate | 1.0 | |
| Distearoylethyl Hydroxyethylmonium Methosulfate | 1.0 | |
| Cetyl Palmitate | 0.5 | |
| Cetearyl alcohol | 5.0 | 3.0 |
| Guar Hydroxypropyltrimonium Chloride | 0.1 | 0.1 |
| Stearamidopropyl Dimethylamine | 0.5 | 0.5 |
| Behentrimonium Chloride | 1.0 | |
| Cetrimonium Chloride | | 1.0 |
| Polyquaternium-37 | 0.2 | 0.2 |
| Amodimethicone | 0.3 | |
| Dimethicone | 1.0 | |
| Isododecane | | 2.0 |
| Myristyl Alcohol | | 3.0 |
| Propanediol-1,2 | | 0.5 |
| Ethanol | 0.35 | 3.0 |
| Water, preservatives, pH adjusters | to 100 | to 100 |

The two hair cleansing agents A and B were applied to identically pre-treated (cleansed and then pre-damaged) strands of hair from the company Kerling International (Backnang, German European natural hair 7/0; batch #04/2010; N74; length: 12 cm; weight: 1 +/−0:05 g) in the usual manner, and the wet combability was then determined.

After three pre-combing strokes the individual strands were combed with slow rotation 10 times using a comb from the company Hercules Sägemann (hard rubber comb, fine-toothed), and the work if the wet combability was measured:

| | applied force in [mJ] |
|---|---|
| Agent as contemplated herein B | 9 |
| Comparison composition A | 9 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent, comprising—in relation to its total weight—
a) from about 0.1 to about 10% by weight of at least one branched and/or linear alkane with 9 to about 17 carbon atoms,
b) from about 2 to about 10% by weight of at least one linear or branched, saturated or unsaturated fatty alcohol with from about 16 to about 22 carbon atoms,
c) from about 2 to about 10% by weight of at least one linear or branched, saturated or unsaturated fatty alcohol with from about 10 to about 14 carbon atoms,
d) from about 0.1 to about 10% by weight of at least one cationic surfactant, comprising a quaternary ammonium compound, and
e) from about 1 to about 10% by weight of at least one linear or branched monovalent alcohol with 2 to 8 carbon atoms.

2. The hair treatment agent according to claim 1, wherein component a) is selected from the group of isodecane, isoundecane, isododecane, isotridecane, isotetradecane, n-decane, n-undecane, n-dodecane, n-tridecane, n-tetradecane and mixtures thereof.

3. The hair treatment agent according to claim 1, wherein component a) is selected from the group of isoundecane, isododecane, isotridecane and mixtures thereof.

4. The hair treatment agent according to claim 1, wherein component a) is selected from the group of n-undecane, n-dodecane, n-tridecane and mixtures thereof.

5. The hair treatment agent according to claim 1, wherein component b) is selected from the group of cetyl alcohol, cetearyl alcohol, stearyl alcohol and mixtures thereof.

6. The hair treatment agent according to claim 1, wherein component c) is selected from the group of lauryl alcohol, tridecyl alcohol, myristyl alcohol and mixtures thereof.

7. The hair treatment agent according to claim 1, comprising, as component d), at least one C₁₆-C₂₄ alkyl trimethyl ammonium salt.

8. The hair treatment agent according to claim 1, wherein component e) is selected from the group of ethanol, propanol, and mixtures thereof.

9. The hair treatment agent according to claim 1, comprising from about 0.1 to about 6% by weight, based on the total weight of the hair treatment agent, of at least one C₃-C₁₂ polyol.

10. The hair treatment agent according to claim 1, comprising from about 0.1 to about 6% by weight, based on the total weight of the hair treatment agent, of
i) at least one—optionally cationized—amidoamine and/or
ii) at least one cationic polymer.

11. The hair treatment agent according to claim 10, comprising, as the at least one—optionally cationized—amidoamine, at least one of the compounds known under the INCI names Brassicamidopropyl Dimethylamine, Stearamidopropyl Dimethylamine, Cocamidopropyl Dimethylamine, Behenamidopropyl Dimethylamine, Isostearamidopropyl Dimethylamine and Bis-Ethyl(isostearylimidazoline) Isostearamide.

12. The hair treatment agent according to claim 10, comprising, as the at least one cationic polymer, at least one of the compounds known under the INCI names Guar Hydroxypropyltrimonium Chloride and Polyquaternium-37.

13. The hair treatment agent according to claim 1, wherein the hair treatment agent is substantially free from silicones.

14. The hair treatment agent according to claim 1, wherein the hair treatment agent has a pH value in the range of from about 2.5 to about 4.5.

15. A method of using a hair treatment agent comprising: applying the hair treatment agent to hair, wherein the hair treatment agent comprises, based on a total weight of the hair treatment agent;
a) from about 0.1 to about 10% by weight of at least one branched and/or linear alkane with 9 to about 17 carbon atoms,
b) from about 2 to about 10% by weight of at least one linear or branched, saturated or unsaturated fatty alcohol with from about 16 to about 22 carbon atoms,
c) from about 2 to about 10% by weight of at least one linear or branched, saturated or unsaturated fatty alcohol with from about 10 to about 14 carbon atoms,
d) from about 0.1 to about 10% by weight of at least one cationic surfactant, comprising a quaternary ammonium compound, and
e) from about 1 to about 10% by weight of at least one linear or branched monovalent alcohol with 2 to 8 carbon atoms.

16. The method of claim 15 further comprising:
improving the combability of the hair and the ease of spreading with the hair treatment agent.

17. The hair treatment agent according to claim 1, wherein component a) is isododecane.

18. The hair treatment agent according to claim 1, wherein component b) is cetearyl alcohol.

19. The hair treatment agent according to claim 1, wherein component c) is myristyl alcohol.

20. The hair treatment agent according to claim 1, wherein:
component a) is selected from the group of n-undecane, n-dodecane, n-tridecane and mixtures thereof, and wherein component a) is present in the hair treatment agent at from about 1.0 to about 6% by weight, based on the total weight of the hair treatment agent;
component b) is cetearyl alcohol, and component b) is present in the hair treatment agent at from about 3.5 to about 6% by weight, based on the total weight of the hair treatment agent;
component c) is myristyl alcohol, and component c) is present in the hair treatment agent at from about 3.5 to about 6% by weight, based on the total weight of the hair treatment agent;
component d) is selected from the group of cetrimonium salts, behentrimonium salts, and combinations thereof, and component d) is present in the hair treatment agent at from about 1.5 to about 6% by weight, based on the total weight of the hair treatment agent; and
component e) is ethanol, and component e) is present in the hair treatment agent at from about 2.5 to about 6% by weight, based on the total weight of the hair treatment agent.
